(19) United States
(12) Patent Application Publication
(10) Pub. No.: US 2005/0182313 A1
(43) Pub. Date: Aug. 18, 2005

(54) Method and apparatus for noise extraction in measurements of electromagnetic activity in biological sources

(76) Inventor: Don M. Tucker, Eugene, OR (US)

Correspondence Address: BIRDWELL & JANKE, LLP, 1100 SW SIXTH AVENUE, SUITE 1400, PORTLAND, OR, 97204, US

(21) Appl. No.: 10/781,032

(22) Filed: Feb. 17, 2004

(51) Int. Cl. A61B 5/05; A61B 5/04

(52) U.S. Cl. 600/409

(57) ABSTRACT

A method and apparatus for noise extraction in measurements of electromagnetic activity in biological sources. A plurality of body sensors are distributed outside the body for sensing the electromagnetic activity. A plurality of reference sensors are also distributed outside the body, corresponding to the body sensors, for sensing environmental noise. Portions of the body sensor outputs that covary with corresponding portions of the reference sensor outputs are determined and subtracted from the respective body sensor outputs. A shield may be disposed between the body sensors and the reference sensors for shielding the reference sensors from the electromagnetic activity. According to one aspect of the invention, the body sensors are primarily responsive to magnetic fields, and the reference sensors are primarily responsive to electric fields. According to another aspect of the invention, the body sensors are primarily responsive to current flows and the reference sensors are primarily responsive to magnetic fields. According to yet another aspect of the invention, the body sensors are primarily responsive to current flows and the reference sensors are primarily responsive to electric fields.

### BACKGROUND OF THE INVENTION

The present invention relates to a method and apparatus for noise extraction in measurements of electromagnetic activity in biological sources. More particularly, the invention relates to noise extraction in EEG (electroencephalography), ECG (electrocardiography) or MEG (magnetoencephalography) measurements, for increasing the capability to localize the sources of the electromagnetic activity.

In both biomedical research and in treatment of disease it is often important to localize the sources of electromagnetic activity inside the body. This typically cannot be done from inside the body. Rather, the locality of a given source inside the body is inferred from the distribution of electromagnetic field at bio-sensors distributed outside of the body. To solve this problem, known in the art as the “inverse problem,” a source and location of the source is first hypothesized, and the field the source would produce at the sensors is calculated and compared to the field actually measured. Inevitably there is variance, so the source properties are varied iteratively to converge on a solution that yields the smallest error. The error can be minimized only within limits, as the sensors do not respond to the field with perfect fidelity, and they are spaced apart from one another so that it is not possible to measure the field at all locations in space. These practical limitations introduce ambiguities in solving the inverse problem; more than one solution may explain the measured results.

An important source of error that limits convergence in the inverse problem is contamination of the output of the bio-sensors by noise. The source produces a “signal,” but the signal is received by the sensors along with other electromagnetic activity. The other electromagnetic activity includes activity produced by other sources (“environmental noise”) as well as activity produced by the sensors themselves (“sensor generated noise”). Environmental noise may arise from electronic equipment, other biological systems, and atmospheric sources. Sensor generated noise includes electrochemical, thermal, and triboelectric noise in both electrodes and leads. Regardless of the source of the noise, it is always desirable, and often it is critical, to maximize the proportion of signal relative thereto.

The prior art has addressed the problem of noise contamination by attempting to minimize it. Environmental noise is minimized by shielding it from the bio-sensors. Typically, for MEG measurements, the room in which the subject, the bio-sensors, the associated electrical apparatus, and one or more operating technicians are disposed is completely shielded (360 degrees around). This approach to the problem has produced less than satisfactory results, and shielding an entire room is expensive and cumbersome. For other types of measurements such as EEG, shielding was previously provided for the entire room but that has become less common. Instead, 60 Hz line filtering has been used even though environmental noise can be both radiated and broad band.

A recent development in the field of MEG frames the problem differently than the prior art described above. Rather than minimizing the noise, such as by shielding it out, the new approach assumes that MEG sensor output will be contaminated with noise. The approach seeks merely to measure this noise and to subtract it from the MEG sensor output to reveal the desired signal. The approach therefore uses an additional set of sensors (“reference sensors”) for measuring noise that are used in combination with the MEG sensors used to measure brain signal.

To measure the noise according to the new approach, it is important to shield the reference sensors from the brain signal; it is not important to shield the MEG sensors from the environmental noise as was the goal in the prior art. Accordingly, the new approach makes use of a shield for containing the brain signal, which is a much easier task than shielding out environmental noise because the brain signal is typically of very low strength and is highly localized. The shield can therefore be less elaborate and expensive than shields used in the prior art.

To subtract the noise determined by the reference sensors from the output of the MEG sensors, a portion of the output of the MEG sensors that covaries with the output of the reference sensors is determined. This portion of the output of the MEG sensors is assumed to be noise and is subtracted from the total output of the MEG sensors.

The approach also recognizes that it is highly desirable to place the reference sensors as close to the associated MEG sensors as possible. In that case, the reference sensors and the MEG sensors will each “see” very nearly the same noise pattern, even though the magnitude of the noise experienced at the different sensors will differ substantially.

To provide for close proximity of the two sets of sensors in MEG measurements, the shield is shaped to fit over and thereby receive the head of an animal subject. It is desirable, though it is not essential, to substantially enclose the head except at the neck of the animal where complete enclosure is not possible. The shield includes adjacent inner and outer chambers. The inner chamber has an inner wall in closest proximity the head and a spaced-apart lead partition wall that forms a partition between the two chambers. The outer chamber has an outer wall spaced apart from the partition wall.

In the first chamber is disposed a plurality of SQUID devices used as MEG brain sensors. In the second chamber is disposed an associated plurality of SQUID devices used as reference sensors. Each chamber also contains a super-cooling fluid, such as liquid helium, suitable for providing and maintaining a super-conducting temperature for the brain sensors, the reference sensors, and the lead partition wall.

The super-conducting lead partition wall provides electromagnetic shielding for preventing brain signals from propagating to the reference sensors. Since the shield (hereinafter termed “SS” for “superconducting shield”) conforms closely to the head, it is relatively small and, therefore, relatively inexpensive as compared to shielding provided for an entire room.

Since the SS does not completely surround the head, some environmental noise can enter the volume inside the shield and reach the brain sensors. However, as pointed out above, this is not a problem since the shield is effective to isolate the reference sensors from brain activity. On the other hand, the brain signal is of too little strength to leak substantially out of the volume inside the shield and propagate to the reference sensors. Therefore, the reference sensors can give a determination of the environmental noise that is not confounded by brain signal, and once the environmental noise is known, it can be extracted from the MEG sensor output to reveal the brain signal.

While the new approach has outstanding advantages as explained above, it has heretofore been limited to MEG. It is therefore a goal of the present invention to expand on the approach.

### SUMMARY OF THE INVENTION

A method and apparatus for noise extraction in measurements of electromagnetic activity in biological sources according to a first aspect of the present invention provides a plurality of body sensors distributed outside the body, proximate one or more of the sources, for sensing the electromagnetic activity. The body sensors produce respective body sensor outputs. A plurality of reference sensors are also distributed outside the body, corresponding to the body sensors, for sensing environmental noise. The reference sensors produce respective reference sensor outputs. Portions of the body sensor outputs that covary with corresponding portions of the reference sensor outputs are determined and subtracted from the respective body sensor outputs.

According to one aspect of the invention, a shield is disposed between the body sensors and the reference sensors for shielding the reference sensors from the electromagnetic activity. The body sensors are primarily responsive to magnetic fields, and the reference sensors are primarily responsive to electric fields.

According to another aspect of the invention, a shield is disposed between the body sensors and the reference sensors for shielding the reference sensors from the electromagnetic activity. The body sensors include electrodes that are primarily responsive to current flows and the reference sensors are primarily responsive to magnetic fields.

According to yet another aspect of the invention, the body sensors include electrodes that are primarily responsive to current flows and the reference sensors are primarily responsive to electric fields.

### BRIEF DESCRIPTION OF THE DRAWINGS

FIG. 1 is schematic view of a prior art super-conducting shield for use in MEG.

FIG. 2 is a schematic view of a sensor apparatus according to a first aspect of the present invention for use in MEG.

FIG. 3 is a schematic view of an alternative embodiment of the sensor apparatus of FIG. 2.

FIG. 4 is a schematic view of a sensor apparatus according to a second aspect of the present invention for use in EEG.

FIG. 5 is a pictorial view of a prior art geodesic sensor net.

FIG. 6 is a schematic view of a sensor apparatus according to a third aspect of the present invention for use in EEG.

### DETAILED DESCRIPTION OF PREFERRED EMBODIMENTS

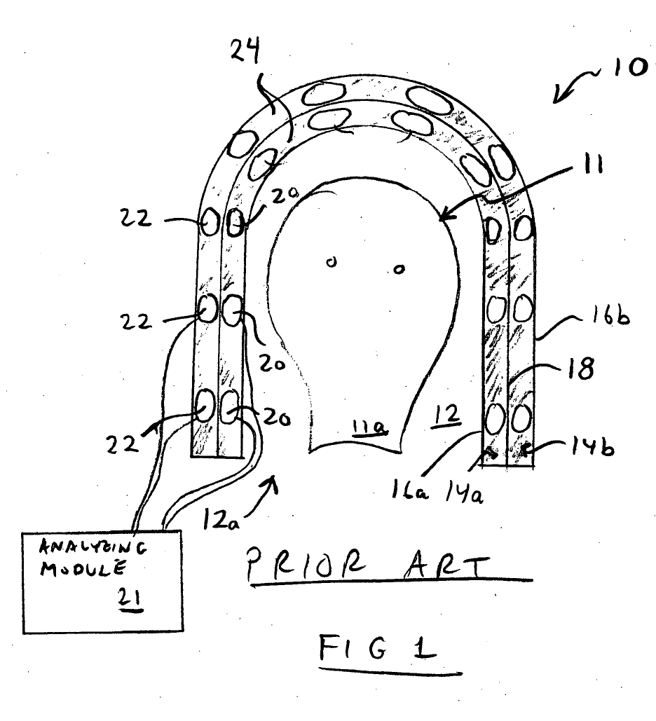

FIG. 1 shows the prior art SS, referenced as 10. The SS 10 has a reentrant volume 12 shaped to fit over and thereby receive the head 11 of an animal subject. The SIS includes adjacent inner and outer chambers 14a and 14b, respectively. The inner chamber 14a has an inner wall 16a in closest proximity to the volume 12 and a spaced-apart lead partition wall 18 that forms a partition between the two chambers. The outer chamber 14b has an outer wall 16b spaced apart from the partition wall 18. The volume 12 has an open end 12a through which extends the neck 11a of the animal subject.

In the first chamber 14a are disposed a plurality of SQUID devices 20 used as MEG brain sensors. In the second chamber 14b are disposed an associated plurality of SQUID devices 22 used as reference sensors. Each chamber also contains a super-cooling fluid 24, such as liquid helium, suitable for providing and maintaining a super-conducting temperature for the brain sensors, the reference sensors, and the lead partition wall.

An analyzing module 21, adapted to receive analog electrical output signals from the MEG reference and brain sensors (only some of the connections are shown for simplicity), convert the signals to digital data and operate on the data is provided for this purpose. The analyzing module typically includes an analog front end for receiving analog SQUID outputs, analog to digital conversion circuitry to produce digital data from the SQUID outputs, and a processor for operating on the digital data. The analyzing module determines a portion of the output of the brain sensors that covaries with the output of the reference sensors and subtracts this covarying portion from the total output of the brain sensors.

As indicated above, the present invention expands on the SIS concept which, while representing a breakthrough in thinking, is limited in scope. According to the present invention, the concept is extended to provide for noise extraction in EEG as well as to provide enhancements for noise extraction in MEG.

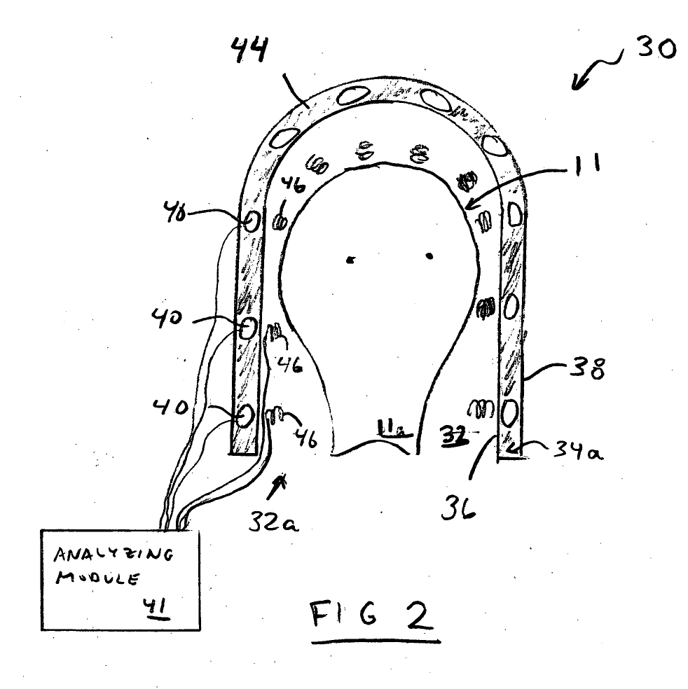

Turning to FIG. 2, a sensor apparatus 30 for enhancing noise extraction in MEG measurements according to the present invention is shown. Like the SS 10, the apparatus 30 has a reentrant volume 32 shaped to fit over and thereby receive the head 11. The volume 32 has an open end 32a through which extends the neck 11a.

The sensor apparatus 30 includes at least one chamber 34a. The chamber 34a has an inner wall 36 in closest proximity to the volume 32 and a spaced-apart outer wall 38. In the chamber 34a are disposed a plurality of SQUID devices 40 used as MEG brain sensors. The chamber also contains a super-cooling fluid 44, such as liquid helium, suitable for providing and maintaining a super-conducting temperature for the brain sensors. Preferably, the outer wall 38 is formed of a material, such as lead, that is superconducting at the temperature of the fluid 44, but it is a recognition in accord with the present invention that this is not essential.

Adjacent the inner wall 36 of the chamber 34a is a plurality of electrically conductive antennae 46 corresponding to the plurality of brain sensors 40. The antennae are preferably formed of metal wire and are electrically insulated from the head of the animal subject, such as by being spaced apart therefrom a small amount. Each of the antennae is preferably disposed as close to a corresponding one of the brain sensors as is practical.

The antennae 46 are preferably shaped so as to be particularly responsive to magnetic fields, e.g., by being wound as coils, but being conductive the antennae 46 are also responsive to current flow. The brain of the animal subject produces electrical activity in the form of currents flowing from sources in the brain to the surface of the skin. However, since the antennae 46 are electrically insulated from the head, they are shielded from current flow, and no significant amounts of electromagnetic energy, including magnetic energy, is radiated from the head. Therefore, the responses of the antennae 46 will be substantially entirely due to environmental noise that leaks into the volume 32 through the open end 32a. This noise can then be extracted from the output of the brain sensors 40.

The electrical outputs of the antennae 46 are susceptible to environmental noise leaking into the volume 32 through the open end 32a, and the antennae are primarily responsive to electrical noise. The SQUID brain sensors 40 are also responsive to environmental noise, but are primarily responsive to magnetic noise. However, it is recognized in accord with the present invention that the magnetic component of the environmental noise is temporally coherent with the electrical component of the environmental noise. Therefore, the pattern of noise determined by the reference sensors 46 covaries with the noise experienced at the brain sensors 40.

Particularly, in a preferred embodiment of the invention, a portion of the output of the brain sensors 40 covaries with the output of the antennae 46. This covarying portion of the output of the brain sensors is assumed to be noise and is subtracted from the total output of the brain sensors. An analyzing module 41, adapted to receive electrical output signals from the reference and brain sensors (only some of the connections are shown for simplicity), convert the signals to digital data and operate on the data is provided for this purpose. The analyzing module typically includes an analog front end for receiving analog SQUID and antenna outputs, analog to digital conversion circuitry to produce digital data from the analog signals, and a processor to operate on the digital data, for analyzing the data. However, the analyzing module may take any form desired to receive sensor signals and perform mathematical or signal processing methods thereon for extracting noise from therefrom that is confounded by noise where the pattern of the noise is known without departing from the principles of the invention.

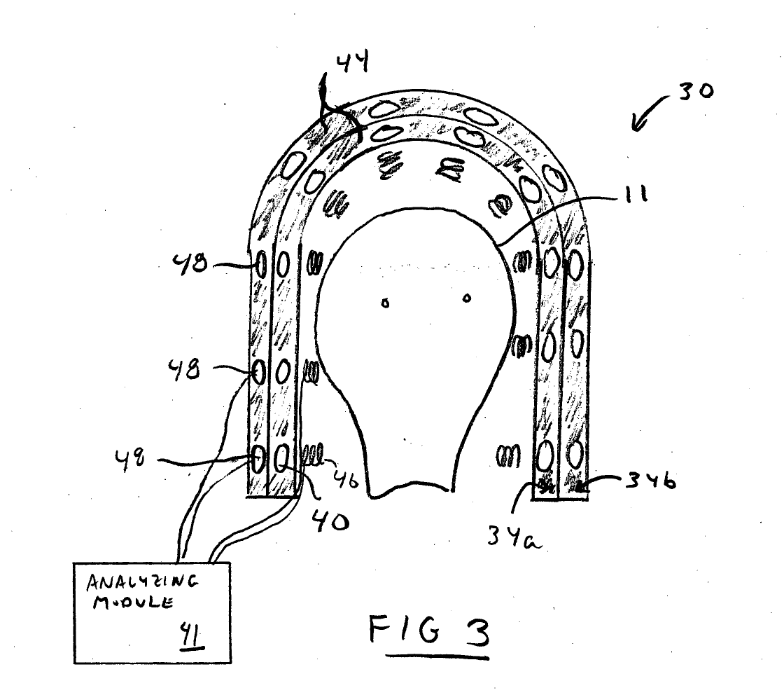

Referring to FIG. 3, the sensor apparatus 30 may include a second chamber 32b, adjacent the chamber 32a, where the chamber 32a is in closest proximity to the volume 32. The chamber 32b also includes the chilling fluid 44, and in the chamber 32b are disposed a plurality of SQUID devices 48 used as magnetic reference sensors. The magnetic reference sensors are, with respect to the interior volume 32, outside the wall 38 and are shielded by the wall from brain signals. The magnetic reference sensors are, therefore, responsive only to environmental noise, are particularly sensitive to magnetic noise, and are used to extract (or cancel) noise from the output of the magnetic brain sensors 40 in the manner described above in connection with the prior art SS 10. The analyzing module 41 is modified accordingly to receive and analyze additional SQUID outputs. The contribution to noise extraction (or cancellation) provided by the antennae 46 is synergistic with that provided by the reference sensors 48 since the antennae 46 have a different noise response than that of the magnetic reference sensors.

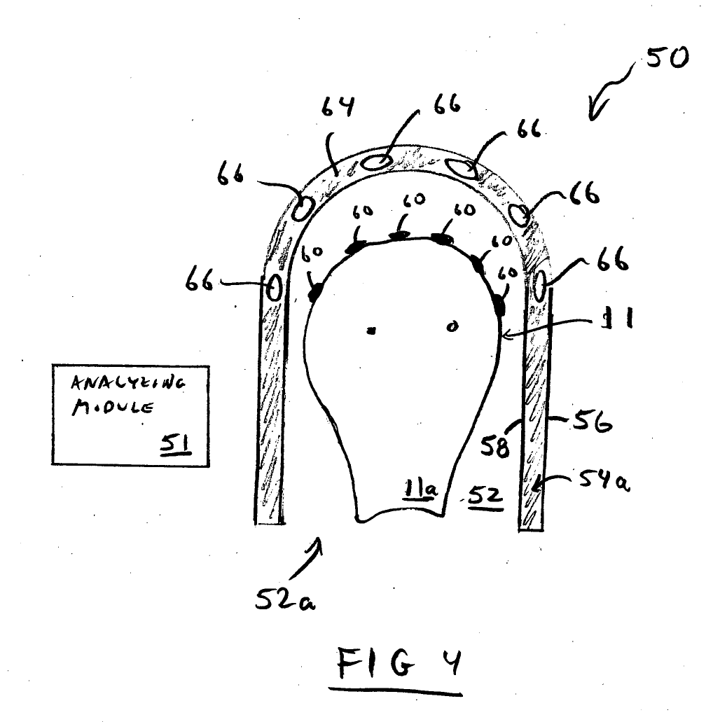

Turning to FIG. 4, a sensor apparatus 50 for use in noise extraction in EEG measurements according to the present invention is shown. Like the SS 10, the apparatus 50 has a reentrant volume 52 shaped to fit over and thereby receive the head 11. The volume 52 has an open end 52a through which extends the neck 11a.

The sensor apparatus 50 includes at least one chamber 54a. The chamber 54a has an inner wall 58 in closest proximity to the volume 52 and a spaced-apart outer wall 56. In the chamber 54a are disposed a plurality of SQUID devices 66 used as MEG reference sensors. The chamber also contains a super-cooling fluid 64, such as liquid helium, suitable for providing and maintaining a super-conducting temperature for the reference sensors. Preferably, the inner wall 58 is formed of a material, such as lead, that is superconducting at the temperature of the fluid 64, but it is a recognition in accord with the present invention that this is not essential. Thence, the requirement for a chamber arises only as a result of the need to keep SQUIDs at superconducting temperatures.

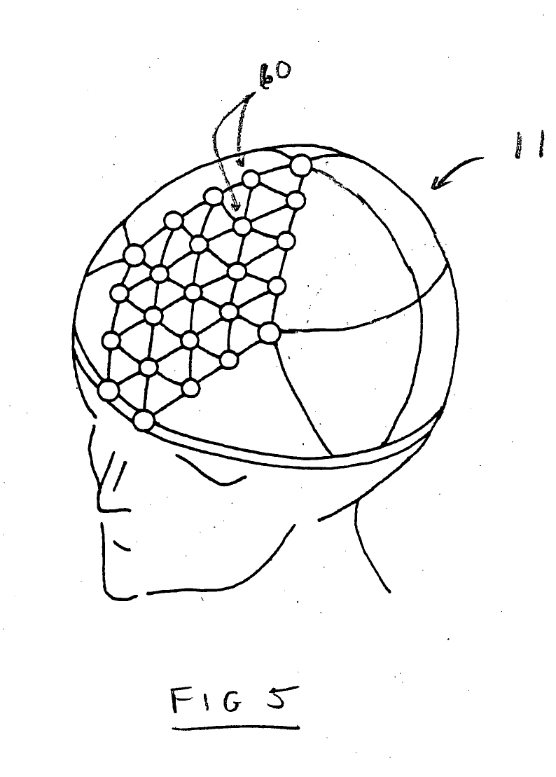

A plurality of electrodes 60 are disposed as brain sensors within the volume 52 for EEG measurements. As mentioned above, the brain of the animal subject produces electrical activity in the form of currents flowing from sources in the brain to the surface of the skin. The electrodes are typically formed of metal plates suitable for making surface contact with the skin and measuring either the current flowing in the skin or the electric potential on the skin responsible for the current by producing a responsive output. Contact of the electrodes with the skin may be enhanced by the use of conductive solutions or gels and/or contact enhancing devices as is known in the art. Referring to FIG. 5, the electrodes 60 are preferably distributed over the surface of the head in a geodesic pattern such as provided by the “geodesic sensor net” described in the inventor's U.S. Pat. No. 5,291,888, but this is not essential. The sensor net connects each of a plurality of electrodes to at least two other electrodes by means of respective flexible members. The relative position of the electrodes is fixed by the flexible members, where each flexible member is under the same amount of tension, in a geodesic pattern.

The electrical outputs of the electrodes 60 are susceptible to environmental noise leaking into the volume 52 through the open end 52a, and the electrodes are primarily responsive to electrical noise. The SQUID reference sensors 66 on the opposite side of the wall 58 are also responsive to environmental noise, but are primarily responsive to magnetic noise. However, as mentioned above, it is recognized in accord with the present invention that the magnetic component of the environmental noise is temporally coherent with the electrical component of the environmental noise. Therefore, the pattern of noise determined by the reference sensors 66 covaries with the noise experienced at the brain sensors 60. Accordingly, the noise can be extracted therefrom as described above. An analyzing module 51 similar to the module 41 described above and adapted for receiving and analyzing SQUID and electrode outputs is provided for this purpose.

Particularly, in a preferred embodiment of the invention, a portion of the output of the brain sensors 60 that covaries with the output of the reference sensors 66 is assumed to be noise and is subtracted from the total output of the brain sensors. It should be understood that any other mathematical or signal processing method for extracting noise from a signal that is confounded by noise where the pattern of the noise is known may be used without departing from the principles of the invention.

In general, according to the invention, reference sensors and brain sensors are preferably provided in closely spaced pairs so that the output of the reference sensor and the output of the corresponding brain sensor can be assumed to covary. This is especially important when using the brain sensors to gather data for solving the inverse problem because the process of finding converging solutions is extremely sensitive to and unstable in the face of noise and other errors. Any displacement between the reference sensor and the brain sensor to which a noise determination made by the reference sensor is applied will generally produce an error. However, it is not essential that there be a 1:1 correspondence between reference and brain sensors so that correspondence may be greater or less than 1:1. As a practical matter, it is often preferable to provide fewer reference sensors to correspond with a greater number of brain sensors because the noise tends to have a relatively constant spatial distribution as compared to the brain signal which requires greater spatial resolution to measure. This reduces cost and complexity without necessarily introducing unacceptable amounts of error. It is also not essential that the reference sensors and brain sensors be spaced any particular maximum distance apart. As will be readily appreciated by persons of ordinary skill, the amount of error in the noise extraction process due to the displacement of a given brain sensor with respect to the reference sensor used to determine the noise therefor will vary depending on the application.

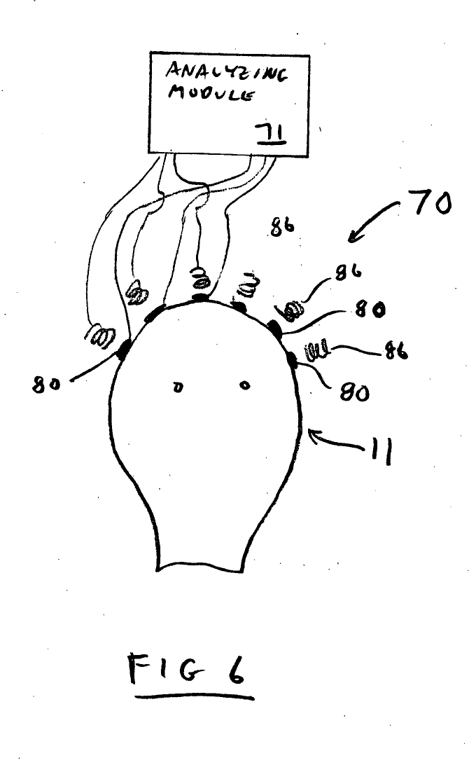

Turning to FIG. 6, a sensor apparatus 70 for noise extraction in EEG measurements according to the present invention is shown. A plurality of electrodes 80 are provided as brain sensors. Like the electrodes 60 described above, the brain sensors 80 are conductive elements that make contact with the skin of the head 11 for conducting and thereby responding to currents flowing therein. Also like the electrodes 60, the brain sensors 80 are preferably distributed over the surface of the head in a geodesic pattern (see FIG. 4) such as described in the aforementioned '888 patent, but this is not essential.

A plurality of antennae 86 are also provided in the sensor net 70 as reference sensors. The reference sensors 86 are physically disposed in close proximity to the corresponding brain sensors 80, however they are electrically insulated therefrom such as by being spaced apart a small amount. Therefore, the reference sensors 86 are shielded from the brain signals produced by sources of electrical activity producing the aforementioned currents.

The reference sensors 86 are shaped so as to be particularly responsive to electric fields of energy radiated within a frequency band that covers the spectrum of environmental noise. The reference sensors 86 are therefore able to measure the environmental noise and, because they are effectively shielded from brain signals, their outputs can be used to extract noise from the brain sensors.

In the sensor apparatus 70, the spacing between the brain and reference sensors may be made small enough that the noise measured by the reference sensors 86 may be simply subtracted from the outputs of the corresponding brain sensors. However, extraction based on the covariance of the outputs for the brain and reference sensors as described above may also be used. An analyzing module 71 similar to the module 41 described above and adapted for receiving and analyzing antenna and electrode outputs and electrode outputs is provided for these purposes.

It is highly desirable to take EEG measurements in an MRI environment, a.k.a. “in the magnet.” However, MRI produces large amounts of electromagnetic noise. For example, RF pulses are used to step through or index the spatial distribution of responses that provide specific image cross-sections. Since a very large magnetic field is used, even very small movements of metal (non-magnetic) electrodes or wires generates current. For example, the small amount of movement of the subject that is induced by blood flow can be sufficient to generate unacceptably high levels of noise. Sensor apparatus according to the present invention, such as the sensor apparatus 70, are particularly well suited to noisy environments such as the MRI, because they do not depend on minimizing or eliminating noise.

It is to be recognized that, while a specific method and apparatus for noise extraction in measurements of electromagnetic activity in biological sources has been shown and described as preferred, other configurations and methods could be utilized, in addition to configurations and methods already mentioned, without departing from the principles of the invention. For example, according to the principles of the invention, apparatus described herein may be modified as appropriate for use with different types of sensors having different physical requirements.

While the invention has been described in a particular form adapted for use in measuring brain activity, the principles of the invention apply equally well to electromagnetic activity in any other part of the body. Where a shield is used, the shield would be suitably modified for such use. For example, where a shield according to the invention is to be used for measurements of heart activity, the shield may be provided in the form of a structure adapted to surround or wrap around the chest.

The terms and expressions which have been employed in the foregoing specification are used therein as terms of description and not of limitation, and there is no intention of the use of such terms and expressions to exclude equivalents of the features shown and described or portions thereof, it being recognized that the scope of the invention is defined and limited only by the claims which follow.

1. An apparatus for increasing the proportion of a responsive signal relative to environmental noise in a system for measuring electromagnetic activity generated by one or more sources inside a living body, comprising:
a plurality of spaced apart body sensors distributed outside the body proximate the one or more sources for sensing the electromagnetic activity, said body sensors being primarily responsive to magnetic fields and producing respective body sensor outputs;
a plurality of spaced apart first reference sensors distributed outside the body corresponding to said body sensors for sensing the environmental noise, said first reference sensors being primarily responsive to electric fields and producing respective first reference sensor outputs;
a shield disposed between said body and said first reference sensors for shielding said first reference sensors from the electromagnetic activity; and
an analyzing module for determining portions of said body sensor outputs that covary with corresponding portions of said first reference sensor outputs and subtracting said portions of said body sensor outputs from the respective said body sensor outputs.

2. The apparatus of claim 1, wherein said body sensors include SQUIDs, further comprising a chamber adapted to contain said SQUIDs immersed in a super-cooling fluid.

3. The apparatus of claim 2, wherein said chamber is adapted to maintain said shield at a super-conducting temperature at the temperature of said fluid.

4. The apparatus of claim 3, wherein said first reference sensors include coils.

5. The apparatus of claim 2, wherein said first reference sensors include coils.

6. The apparatus of claim 1, wherein said first reference sensors include coils.

7. The apparatus of claim 1, further comprising a plurality of spaced apart second reference sensors distributed outside the body, corresponding to said body sensors, for sensing the environmental noise, said second reference sensors being primarily responsive to magnetic fields and producing respective first reference sensor outputs, wherein said shield shields said second reference sensors from the electromagnetic activity.

8. The apparatus of claim 7, wherein said body sensors and said second reference sensors include SQUIDs, further comprising respective chambers each adapted to contain the respective said SQUIDs immersed in a super-cooling fluid.

9. The apparatus of claim 8, wherein said chamber is adapted to maintain said shield at a super-conducting temperature at the temperature of said fluid.

10. The apparatus of claim 9, wherein said first reference sensors include coiled wire.

11. The apparatus of claim 8, wherein said first reference sensors include coiled wire.

12. The apparatus of claim 7, wherein said first reference sensors include coiled wire.

13. An apparatus for increasing the proportion of a responsive signal relative to environmental noise in a system for measuring electromagnetic activity generated by one or more sources inside a living body, comprising:
a plurality of spaced apart body sensing electrodes for surface contact with the body proximate the one or more sources for sensing the electromagnetic activity, said electrodes being primarily responsive to current flows on the surface of the body and producing respective electrode outputs;
a plurality of spaced apart reference sensors distributed outside the body corresponding to said electrodes for sensing the environmental noise, said reference sensors being primarily responsive to magnetic fields and producing respective reference sensor outputs;
a shield disposed between said electrodes and said reference sensors for shielding said reference sensors from the electromagnetic activity; and
an analyzing module for determining portions of said electrode outputs that covary with corresponding portions of said reference sensor outputs and subtracting said portions of said electrode outputs from the respective said body sensor outputs.

14. The apparatus of claim 6, wherein said reference sensors include SQUIDs, further comprising a chamber adapted to contain said SQUIDs immersed in a super-cooling fluid.

15. The apparatus of claim 14, wherein said chamber is adapted to maintain said shield at a super-conducting temperature at the temperature of said fluid.

16. The apparatus of claim 15, further comprising a sensor net adapted to connect each of said electrodes to at least two others of said electrodes by means of respective flexible members.

17. The apparatus of claim 16, wherein said sensor net is adapted to fix the relative positions of said electrodes, where each of said flexible members is under the same amount of tension, in a geodesic pattern.

18. The apparatus of claim 14, further comprising a sensor net adapted to connect each of said electrodes to at least two others of said electrodes by means of respective flexible members, wherein said sensor net is adapted to fix the relative positions of said electrodes, where each of said flexible members is under the same amount of tension, in a geodesic pattern.

19. The apparatus of claim 13, further comprising a sensor net adapted to connect each of said electrodes to at least two others of said electrodes by means of respective flexible members, wherein said sensor net is adapted to fix the relative positions of said electrodes, where each of said flexible members is under the same amount of tension, in a geodesic pattern.

20. An apparatus for increasing the proportion of a responsive signal relative to environmental noise in a system for measuring electromagnetic activity generated by one or more sources inside a living body, comprising:
a plurality of spaced apart body sensing electrodes for surface contact with the body proximate the one or more sources for sensing the electromagnetic activity, said electrodes being primarily responsive to current flows on the surface of the body and producing respective electrode outputs;
a plurality of spaced apart reference sensors distributed outside the body corresponding to said electrodes for sensing the environmental noise, said reference sensors being primarily responsive to electric fields and producing respective reference sensor outputs; and
an analyzing module for determining portions of said body sensor outputs that covary with corresponding portions of said reference sensor outputs and subtracting said portions of said body sensor outputs from the respective said body sensor outputs.

21. The apparatus of claim 20, further comprising a sensor net adapted to connect each of said electrodes to at least two others of said electrodes by means of respective flexible members.

22. The apparatus of claim 21, wherein said sensor net is adapted to fix the relative positions of said electrodes, where each of said flexible members is under the same amount of tension, in a geodesic pattern.

23. A method for increasing the proportion of a responsive signal relative to environmental noise in a system for measuring electromagnetic activity generated by one or more sources inside a living body, comprising:
distributing a plurality of spaced apart body sensors outside the body proximate the one or more sources, said body sensors being primarily responsive to magnetic fields;
sensing the electromagnetic activity with said body sensors and producing respective body sensor outputs representative thereof;
distributing a plurality of spaced apart first reference sensors outside the body corresponding to said body sensors, said first reference sensors being primarily responsive to electric fields;
sensing the environmental noise with said reference sensors and producing respective first reference sensor outputs representative thereof;
shielding said first reference sensors from the electromagnetic activity;
determining portions of said body sensor outputs that covary with corresponding portions of said first reference sensor outputs; and
subtracting said portions of said body sensor outputs from the respective said body sensor outputs.

24. The method of claim 23, wherein said body sensors include SQUIDs.

25. The method of claim 24, wherein said shield is super-conducting.

26. The method of claim 23, wherein said shield is superconducting.

27. The method of claim 23, further comprising distributing a plurality of spaced apart second reference sensors outside the body corresponding to said body sensors, said second reference sensors being primarily responsive to magnetic fields, shielding said second reference sensors from the electromagnetic activity, sensing the environmental noise and producing respective first reference sensor outputs representative thereof.

28. The method of claim 27, wherein said body sensors and said second reference sensors include SQUIDs.

29. The method of claim 28, wherein said step of shielding includes providing a super-conducting shield.

30. The method of claim 27, wherein said step of shielding includes providing a super-conducting shield.

31. A method for increasing the proportion of a responsive signal relative to environmental noise in a system for measuring electromagnetic activity generated by one or more sources inside a living body, comprising:
distributing a plurality of spaced apart body sensing electrodes for surface contact with the body proximate the one or more sources, said electrodes being primarily responsive to current flows on the surface of the body;
sensing the electromagnetic activity with said electrodes and producing respective electrode outputs representative thereof;
distributing a plurality of spaced apart reference sensors distributed outside the body corresponding to said electrodes, said reference sensors being primarily responsive to magnetic fields;
sensing the environmental noise and producing respective reference sensor outputs representative thereof;
shielding said reference sensors from the electromagnetic activity;
determining portions of said electrode outputs that covary with corresponding portions of said reference sensor outputs; and
subtracting said portions of said electrode outputs from the respective said body sensor outputs.

32. The method of claim 31, wherein said reference sensors include SQUIDs.

33. The method of claim 32, wherein said step of shielding includes providing a super-conducting shield.

34. The method of claim 31, wherein said step of shielding includes providing a super-conducting shield.

35. The method of claim 34, further comprising connecting each of said electrodes to at least two others of said electrodes by means of respective flexible members.

36. The method of claim 35, further comprising fixing the relative positions of said electrodes, where each of said flexible members is under the same amount of tension, in a geodesic pattern.

37. The method of claim 33, further comprising connecting each of said electrodes to at least two others of said electrodes by means of respective flexible members, and fixing the relative positions of said electrodes, where each of said flexible members is under the same amount of tension, in a geodesic pattern.

38. The method of claim 32, further comprising connecting each of said electrodes to at least two others of said electrodes by means of respective flexible members, and fixing the relative positions of said electrodes, where each of said flexible members is under the same amount of tension, in a geodesic pattern.

39. The method of claim 31, further comprising connecting each of said electrodes to at least two others of said electrodes by means of respective flexible members, and fixing the relative positions of said electrodes, where each of said flexible members is under the same amount of tension, in a geodesic pattern.

40. A method for increasing the proportion of a responsive signal relative to environmental noise in a system for measuring electromagnetic activity generated by one or more sources inside a living body, comprising:
distributing a plurality of spaced apart body sensing electrodes for surface contact with the body proximate the one or more sources, said electrodes being primarily responsive to current flows on the surface of the body;
sensing the electromagnetic activity with said body sensing electrodes and producing respective electrode outputs representative thereof;
a plurality of spaced apart reference sensors distributed outside the body corresponding to said electrodes for sensing the environmental noise, said reference sensors being primarily responsive to electric fields and producing respective reference sensor outputs;
sensing the environmental noise with said reference sensors and producing respective electrode outputs representative thereof;
determining portions of said electrode outputs that covary with corresponding portions of said reference sensor outputs; and
subtracting said portions of said electrode outputs from the respective said body sensor outputs.

41. The method of claim 40, further comprising connecting each of said electrodes to at least two others of said electrodes by means of respective flexible members.

42. The method of claim 41, further comprising fixing the relative positions of said electrodes, where each of said flexible members is under the same amount of tension, in a geodesic pattern.
